(19) United States
(12) Patent Application Publication
(10) Pub. No.: US 2025/0049737 A1
(43) Pub. Date: Feb. 13, 2025

(54) Lauflumide and derivatives thereof for treating chronic fatigue syndrome and myalgic encephalomyelitis/chronic fatigue syndrome (ME/CFS)

(71) Applicant: NLS Pharmaceutics AG, Zurich (CH)

(72) Inventors: Eric KONOFAL, Senlis (FR); Emily Samuela TURILLI, Lecco (IT)

(73) Assignee: NLS Pharmaceutics AG, Zurich (CH)

(21) Appl. No.: 18/576,881

(22) PCT Filed: Jul. 8, 2022
(86) PCT No.: PCT/EP2022/069170

§ 371 Date: Jan. 5, 2024

(30) Foreign Application Priority Data
Jul. 8, 2021 (EP) 21305944.7

(51) Int. Cl. A61K 31/165; A61P 43/00

(52) U.S. Cl. CPC A61K 31/165; A61P 43/00

(57) ABSTRACT

The invention concerns a compound of formula (I), wherein R1=halogen atom selected in the group consisting of: F, Cl, Br, I, and R2=H or OH, or a pharmaceutically acceptable isomer, salt and/or solvate thereof, for use in preventing and/or treating chronic fatigue syndrome.

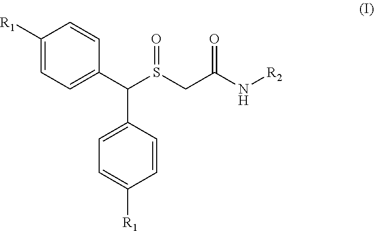

### FIELD OF THE PRESENT INVENTION

The present invention relates to the use of Lauflumide and derivatives thereof in the prevention and/or treatment of chronic fatigue syndrome, especially when it is due to the COVID-19 disease due to SARS-Cov2 virus.

### BACKGROUND OF THE PRESENT INVENTION

The global confirmed case count of coronavirus disease 2019 (COVID-19) surpassed 534 million as of June 2022 (Coronavirus disease (COVID-19), 2022) with more than 30% of individuals affected by COVID-19, including asymptomatic cases may experience post-COVID neuropsychiatric late sequelae¹⁻⁵.

Fatigue, sleep disorders (e.g. circadian rhythm disorders), cognitive impairment, along with other enduring neuropsychiatric (e.g., anxiety, depression), colloquially referred to as ‘long COVID’⁶⁻⁹.

The National Institute for Health and Care Excellence (NICE) defines ‘post-COVID-19 syndrome’ (PCS) as a constellation of symptoms which develop during or following COVID-19 infection, persist for >12 weeks, and are not sufficiently explained by alternative diagnoses (https://www.nice.org.uk/guidance/ng188).

Post COVID-19 condition is defined as persistent symptoms usually occurring 3 months from onset in individuals with past confirmed or probable SARS-CoV-2 infection and persisting for at least 2 months which cannot be explained by an alternative diagnosis (https://www.who.int/publications/i/item/WHO-2019-nCoV-Post_COVID-19_condition-Clinical_case_definition-2021.1).

Fatigue, cognitive impairment and sleep disorders have been consistently reported to be some of the most common and debilitating features of PCS and constituting a significant global economic burden, respectively^{7,8,10,11}. Unlike other common symptoms of PCS including dyspnea and depression, there are no established and effective treatments for post-viral fatigue and cognitive impairment, as well as related conditions such as Myalgic Encephalomyelitis/Chronic Fatigue Syndrome (ME/CFS)¹².

ME/CFS has been identified in the 1980s. This syndrome is characterized by an extreme fatigue and a pain felt in routine activities, post-exertional malaise and unrefreshing sleep. Other secondary symptoms may also occur such as impaired memory or ability to concentrate and orthostatic intolerance.

According to Fukuda's criteria¹³, as modified according to Reeves, et al¹⁴, chronic fatigue syndrome (CFS) is an illness characterized by 1) the presence of persisting, debilitating fatigue that does not resolve with bed rest, and lasts for at least six months resulting in severe impairment in daily function; 2) by symptoms and disability that cannot be ascribed to any other medical and psychiatric conditions. Diagnosis relies in large part on behavioural markers, either patients' self-reported symptoms or observations by clinicians. Laboratory diagnostic and screening tests are not widely available.

CFS is responsible for significant morbidity and occurs in an estimated 0.42% of people, predominately female, in the United States and worldwide. Prior work strongly suggests this complex, multi-symptom illness has a multi-system pathogenesis that involves the nervous, endocrine and immune systems. Abnormalities in stress responses have been identified as potential triggers or mediators of CFS symptoms.

Between 836,000 and 2.5 M Americans suffer from ME/CFS. Direct and indirect economic costs to society are estimated between $17 and $24 billion each year¹⁵.

The etiology of ME/CFS is principally post-viral. Acute viral infections and subsequent overlapping immune responses are established triggers for the onset of ME/CFS symptoms. It has been shown that prior viral epidemics (SARS virus, MERS virus) led to increased ME/CFS occurrence as long-term consequences^{12,16,17}.

There are still no effective and safe therapies up to now; no FDA approved drugs for post-COVD or Long COVID sequelae (e.g. fatigue, cognitive impairment, circadian rhythm disorders)^{18,19}.

The only double-blind RCT of a CNS drug to provide statistically significant improvement in fatigue score and concentration was using methylphenidate. However, given the significant adverse effects and potential for dependence associated with this drug, it is not an ideal choice for the treatment of ME/CFS¹⁹.

Xyrem (sodium oxybate, SXB) as well shows severe side effects while not being particularly effective. The FDA declared that a dangerous drug such as SXB cannot be approved for indications involving large populations (such as ME/CFS or fibromyalgia), as this would favour illicit use as a rape drug and recreational substance²⁰.

Jazz Pharmaceuticals is exploring the field of ME/CFS: as post-marketing data and patients reports for Sunosi® (solriamfetol) showed poor efficacy on Narcolepsy, Jazz is now looking at ME/CFS as a new indication: an 8-week single center, randomized, double-blind, placebo-controlled, flexible titration Phase 4 clinical trial with solriamfetol started on April 1^{st}, involving 44 adult subjects²¹.

Neuropeptide Y (NPY) is found in both the central and peripheral nervous systems. In the peripheral nervous system, NPY is concentrated in and released from sympathetic nerve endings, either alone or with catecholamines. NPY release follows stress such as strenuous exercise, panic disorders, and cold exposure. In the periphery, NPY is activating and stimulates the stress response, but in the brain, NPY is anxiolytic, inhibitory of sympathetic activity and causes lowering of blood pressure and heart rate. Concentrations of NPY in the brain are higher than other neuropeptides, particularly in the limbic system, including the amygdala and the hypothalamus, all areas of the brain involved with emotion.

NPY has been found to be a useful biomarker for CFS²². Plasma NPY was elevated in CFS subjects, compared to controls (p=0.000) and to GWI (Gulf War Illness) cases (p=0.000). Receiver operating characteristics (ROC) curve analyses indicated that the predictive ability of plasma NPY to distinguish CFS patients from healthy controls and from GWI was significantly better than chance alone. In 42 patients with CFS, plasma NPY had significant correlations (<0.05) with perceived stress, depression, anger/hostility, confusion, negative thoughts, positive thoughts, general health, and cognitive status. In each case the correlation (+ or −) was in the anticipated direction. This study reports that plasma NPY is elevated compared to healthy controls and to a fatigued comparison group, GWI patients. The significant correlations of NPY with stress, negative mood, general health, depression and cognitive function strongly suggest that this peptide be considered as a biomarker to distinguish subsets of CFS.

Even mild agonist reverse inhibitor effect on NPY1 may be of relevance in mood disorders treatment²³ and histamine H2 receptor stimulation improved retention of the memory associated with a contextual fear conditioning procedure in rats²⁴ as enhanced memory consolidation of inhibitory avoidance learning in mice^{25,26}.

Lauflumide [2-((bis(4-fluorophenyl)methyl)sulfinyl)acetamide] is a next-generation of selective dopamine (DA) reuptake inhibitor, an enantiomeric form (R) optically pure with an enantiomeric excess of more than 95% of bis (p-fluoro)phenyl ring-substituted derivative of modafinil (USPTO Patent Application 20130295196, Lauflumide and the enantiomers thereof, method for preparing same and therapeutic uses thereof, issued).

Lauflumide is a highly potent wake-promoting agent with no sign of hypersomnia rebound, a potent stimulant with different effects than primary modafinil parent analogues investigated so far²⁷ and most other stimulants in terms of potency and effects on effectiveness sleep rebound and the sleep spectral analysis²⁸. As compared to modafinil, recovery sleep after Lauflumide treatment is characterized by fewer non-rapid eye movement (NREM) amount and delta activity, suggesting a lower need for recovery despite longer drug-induced wakefulness²⁸.

Lauflumide as a wake-promoting agent with a potential cognitive benefit and anti-impulsive effects, shows a lower risk of side effects such as arterial hypertension, or sleep rebound, and could be useful in the treatment of ADHD and arousal-related disorders (e.g. narcolepsy with or without cataplexy and idiopathic hypersomnia)²⁸.

As opposed to modafinil and most of wakefulness agents, Lauflumide fails to stimulate the norepinephrine (NE) sympathetic nervous system leading to deleterious effects on cardiovascular system with a potent arterial hypertension at long-term processing.

Modafinil is weakly DAT inhibitor compared to Lauflumide. Above all, modafinil is thought to be dependent on catecholaminergic (DA and NE) signalling for its wake-promoting effects^{27,29,30}.

Past and recent studies of modafinil analogues demonstrated that DAT inhibition did not correlate with wakefulness-promoting effects in animals, and a number of analogues without any significant inhibition of the DAT still produced wakefulness-promoting effects^{31,32}.

Another study found that modafinil has a long duration of action, with its wake promoting properties largely arising from dopaminergic activity³³.

Other possible mechanisms for the wakefulness effect besides the DAT inhibitor include the indirect activation of orexin systems^{34,36}.

Concerning behavioural data, it has been reported that modafinil (128 mg/kg) strongly reinstated cocaine conditioned place preference following extinction in rats, suggesting that modafinil may induce relapse or increase the vulnerability of addicts to the reinforcing effects of environmental triggers^{37,38}, when the benefit on impulsivity has not been demonstrated. Impulsive choices on a decision-making task in ambiguous situation in patients with narcolepsy, when psychostimulants fail to have an effect on that, remain troublesome³⁹.

Surprisingly, the inventors observed that Lauflumide is a reverse agonist inhibitor of NPY1.

Indeed, Lauflumide is found to weakly bind with neuropeptide Y1 (NPY1), as a reverse agonist inhibitor (27.3%), and possibly exert some of a histaminergic H2 receptor-mediated inducer (>40%) when it was tested at 1.0×10-05 M and that its binding property was calculated as a percent inhibition of the binding of a radioactively labelled ligand specific for each target in accordance with Eurofins validation Standard Operating Procedure (Study 100014859 Eurofins CEREP 20/03/14, unpublished data).

Previous findings have implicated the relaxin-3/RXFP3 system in control of pain transmission, providing new opportunities for the development of therapeutic tools for pain management, by targeting a neuropeptide system that impacts several behaviours that are altered in chronic pain conditions (e.g. fibromyalgia).

Other findings have suggested relaxin-3/RXFP3 signaling in key hypothalamic and limbic circuits is capable of integrating stress-related external and internal information, by regulating the networks responsible for orexigenic and goal-directed (motivated) behaviors⁴⁰.

On circadian rhythm and arousal, the relaxin-3/RXFP3 signaling promote a range of consummatory behaviors is in line with its likely primary role in driving arousal and motivated behavior more broadly^{41,42}.

The inventors postulate that Relaxin-3/RXFP3 signaling was involved in ME/CFS circuit via DAT inhibition.

RXFP3 influencing neuroendocrine control of metabolism, it is highlighted that Lauflumide targeting on RXFP3 receptors may potentially treated feeding-related disorders⁴³.

Lauflumide is a strong DAT inhibitor, and improved impulsivity at the three doses tested with a better significant response than modafinil.

Lauflumide acting on RXFP3 receptors (23.9%) and as a DAT reuptake inhibitor (84.4%) may explain why Lauflumide do not lead to hypersomnolence rebound compared to modafinil in same experimental conditions²⁸.

As an agonist reverse inhibitor effect based on NPY1 receptors, Lauflumide may be of relevance in mood disorders treatment and also acting on histamine H2 receptor may improve retention of the memory associated with a contextual fear and also acting on RXFP3 receptor and DAT inhibition may improve arousal and motivated behavior as impulsivity.

Lauflumide is considered as a potential candidate for post-COVID-19 fatigue pharmacotherapy.

Lauflumide as a wake-promoting agent with a potential cognitive benefit and anti-impulsive effects, shows a lower risk of side effects such as arterial hypertension, or sleep rebound, and could be useful in those with an arousal-related disorders and CFS subsequent to COVID-19 infection (post-COVID syndrome, long COVID).

### SUMMARY OF THE PRESENT INVENTION

An object of the invention is a compound of formula (I)

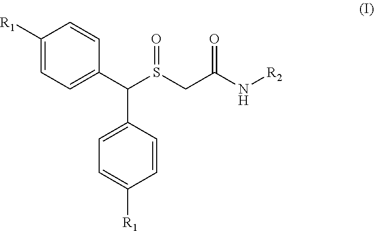

wherein R1=halogen atom selected in the group consisting of: F, Cl, Br, I, and R2=H or OH, or a pharmaceutically acceptable isomer, salt and/or solvate thereof, for use in preventing and/or treating chronic fatigue syndrome.

Another object of the invention is a pharmaceutical composition comprising a compound of formula (I) or a pharmaceutically acceptable isomer, salt and/or solvate thereof and a pharmaceutically acceptable excipient for use in preventing and/or treating chronic fatigue syndrome.

### FIGURES

FIG. 1 Distance travelled by Sham and Control groups (in cm; mean+SEM) during the full 24-h period on days 1, 2 and 3. Difference vs. Sham group: * p≤0.05 or less.

FIG. 2 Distance travelled by Sham and Control groups (in cm; mean+SEM) during the full 24-h period on days 1, 2 and 3 (dark+light phases). Difference vs. Sham group: * p≤0.05 or less.

FIG. 3 Difference of distance travelled by Sham and Control groups (in cm; mean+SEM) between the dark phase and the light phase on days 1, 2 and 3. Difference vs. Sham group: * p≤0.05 or less.

FIG. 4 Distance travelled by Sham and Control groups (in cm; mean±SEM) during the 2-h periods of the dark and light phases on days 1, 2 and 3. For each 12-h period, differences Sham group vs. Control group: G=group effect, G×P=group×period interaction; ns=not significant, * p≤0.05 or less.

FIG. 5 Distance travelled by Sham, Control and Modafinil groups (in cm; mean+SEM) during the 12-h dark and light phases on days 1, 2 and 3. Differences vs. Sham group: * p≤0.05 or less; vs. Control group: #p≤0.05 or less.

FIG. 6 Distance travelled by Sham, Control and Modafinil groups (in cm; mean+SEM) during the full 24-h period on days 1, 2 and 3 (dark+light phases). Differences vs. Sham group: * p≤0.05 or less; vs. Control group: #p≤0.05 or less.

FIG. 7 Difference of distance travelled by Sham, Control and Modafinil groups (in cm; mean+SEM) between the dark phase and the light phase on days 1, 2 and 3. Differences vs. Sham group: * p≤0.05 or less; vs. Control group: #p≤0.05 or less.

FIG. 8 Distance travelled by Sham, Control and Modafinil 32 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less

FIG. 9 Distance travelled by Sham, Control and Modafinil 64 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 10 Distance travelled by Sham, Control and Modafinil 128 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 11 Distance travelled by Sham, Control and NLS-4 groups (in cm; mean+SEM) during the 12-h dark and light phases on days 1, 2 and 3. Differences vs. Sham group: * p≤0.05 or less; vs. Control group: #p≤0.05 or less.

FIG. 12 Distance travelled by Sham, Control and NLS-4 groups (in cm; mean+SEM) during the full 24-h period on days 1, 2 and 3. Differences vs. Sham group: * p≤0.05 or less; vs. Control group: #p≤0.05 or less.

FIG. 13 Difference of distance travelled by Sham, Control and NLS-4 groups (in cm; mean+SEM) between the dark phase and the light phase on days 1, 2 and 3. Differences vs. Sham group: * p≤0.05 or less; vs. Control group: #p≤0.05 or less.

FIG. 14 Distance travelled by Sham, Control and NLS-4 16 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 15 Distance travelled by Sham, Control and NLS-4 32 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 16 Distance travelled by Sham, Control and NLS-4 64 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 17 Distance travelled by Sham, Control and NLS-4 128 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 18 Distance travelled by Sham, Control and NLS-4 256 groups (in cm; mean±SEM) during the 2-h periods of the dark and light phases on days 1, 2 and 3. For each 12-h period, differences vs. Sham group and vs. Control group: G=group effect, G×P=group×period interaction; ns=not significant, * p≤0.05 or less.

FIG. 19 Distance travelled by Sham, Control, Modafinil and NLS-4 groups (in cm; mean+SEM) during the 12-h dark and light phases on days 1, 2 and 3. Differences vs. Sham group * p≤0.05 or less; vs. Control group: #p≤0.05 or less.

FIG. 20 Distance travelled by Sham, Control, Modafinil and NLS-4 groups (in cm; mean+SEM) during the full 24-h period on days 1, 2 and 3. Differences vs. Sham group: * p≤0.05 or less; vs. Control group: #p≤0.05 or less.

FIG. 21 Difference of distance travelled by Sham, Control, Modafinil and NLS-4 groups (in cm; mean+SEM) between the dark phase and the light phase on days 1, 2 and 3. Differences vs. Sham group: * p≤0.05 or less; vs. Control group: #p≤0.05 or less.

FIG. 22 Distance travelled by Sham, Control, Modafinil 32 and 64 and NLS-4 16 groups (in cm; mean+SEM) during the 12-h dark and light phases on days 1, 2 and 3. Differences vs. Sham group: * p≤0.05 or less; vs. Control group: #p≤0.05 or less; vs. Modafinil 32 group: & p≤0.05 or less; vs. Modafinil 64 group: § p≤0.05 or less.

FIG. 23 Distance travelled by Sham, Control, Modafinil 32 and 64 and NLS-4 16 groups (in cm; mean+SEM) during the full 24-h period on days 1, 2 and 3. Differences vs. Sham group: * p≤0.05 or less; vs. Control group: #p≤0.05 or less; vs. Modafinil 32 group: & p≤0.05 or less; vs. Modafinil 64 group: § p≤0.05 or less

FIG. 24 Difference of distance travelled by Sham, Control, Modafinil 32 and 64 and NLS-4 16 groups (in cm; mean+SEM) between the dark phase and the light phase on days 1, 2 and 3. Differences vs. Sham group: * p≤0.05 or less; vs. Control group: #p≤0.05 or less; vs. Modafinil 32 group: & p≤0.05 or less; vs. Modafinil 64 group: § p≤0.05 or less.

FIG. 25 Distance travelled (in cm; mean±SEM) during the 2-h periods of the dark and light phases on days 1, 2 and 3 by Sham, Control, Modafinil 32 (upper panel), Modafinil 64 (lower panel) and NLS-4 16 groups. For each 12-h period, differences NLS-4 16 group vs. Sham, Control, Modafinil 32 and Modafinil 64 groups: G=group effect, G×P=group×period interaction; ns=not significant, * p≤0.05 or less.

FIG. 26 Body weight (in g; mean, SEM) before the beginning and during the fatigue procedure (F-D1-7) and during the test period (T-D1-D3+24 h).

### DETAILED DESCRIPTION OF THE PRESENT INVENTION

The first subject-matter of the invention relates to a compound of formula (I)

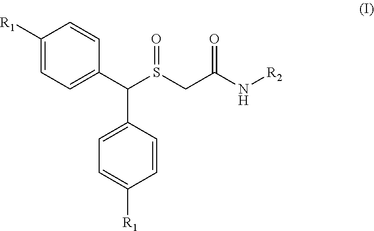

wherein R1=a halogen atom selected in the group consisting of: F, Cl, Br, I, preferably F, and R2=H or OH, preferably R2=H,
or a pharmaceutically acceptable isomer, salt and/or solvate thereof, for use in preventing and/or treating chronic fatigue syndrome (CFS).

Formula (I) has a chiral center at the sulfoxide (—S═O) group.

Thus, “isomer” means preferably “enantiomer”.

According to the present invention, and when not specified otherwise, the term “compound of formula (I)” refers to compound of formula (I) in its racemic form or in its enantiomeric forms.

An “optically pure compound of formula (I)” means an enantiomer in an enantiomeric excess of more than 95%, preferably of more than 96%, more preferably of more than 97%, even more preferably of more than 98%, particularly preferably of more than 99%.

Preferably, R1=F and R2=H.

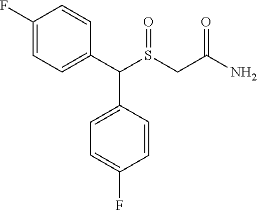 [SMILES: NC(=O)CS(=O)C(c1ccc(F)cc1)c1ccc(F)cc1]

When R1=F and R2=H, optically pure R-enantiomer, compound of formula (I) is Lauflumide, 2-[(R)-((bis(4-fluorophenyl)methane)sulfinyl)]acetamide, and its salts

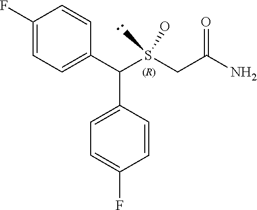

Preferably, the subject-matter of the invention is a compound of formula (I) for use in preventing and/or treating a post-viral infection CFS, more preferably a post-SARS-Cov2 infection CFS, i.e. the long COVID condition.

Compound of formula (I) is preferably used at a therapeutic dose comprised between 50 mg/day and 500 mg/day, more preferably between 80 and 320 mg/day.

Compound of formula (I) is preferably used at a therapeutic dose comprised between 1 mg/kg/day and 5 mg/kg/day.

The second subject-matter of the invention relates to a method of prevention and/or treatment of chronic fatigue syndrome comprising the administration of a compound of formula (I)

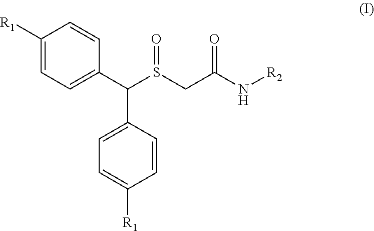

wherein R1=H or a halogen atom selected in the group consisting of: F, Cl, Br, I, preferably F, and R2=H or OH,
or a pharmaceutically acceptable isomer, salt and/or solvate thereof, to a patient in need thereof.

The third subject-matter of the invention relates to a pharmaceutical composition comprising a compound of formula (I) or a pharmaceutically acceptable isomer, salt and/or solvate thereof as defined above and a pharmaceutically acceptable excipient for use in preventing and/or treating chronic fatigue syndrome.

The pharmaceutical composition for use according to the invention is preferably suitable for oral or parenteral administration.

Preferably, the pharmaceutical composition for use according to the invention is suitable for oral administration, for example in the form of a tablet, a capsule, a syrup, a solution, a powder or parenteral administration, for example in the form of a solution, such as an injectable solution or a Transdermal Delivery System (TDS).

The fourth subject-matter of the invention relates to a method of prevention and/or treatment of chronic fatigue syndrome comprising the administration of a pharmaceutical composition as defined above to a patient in need thereof.

### Examples

### Synthesis Route of Lauflumide:

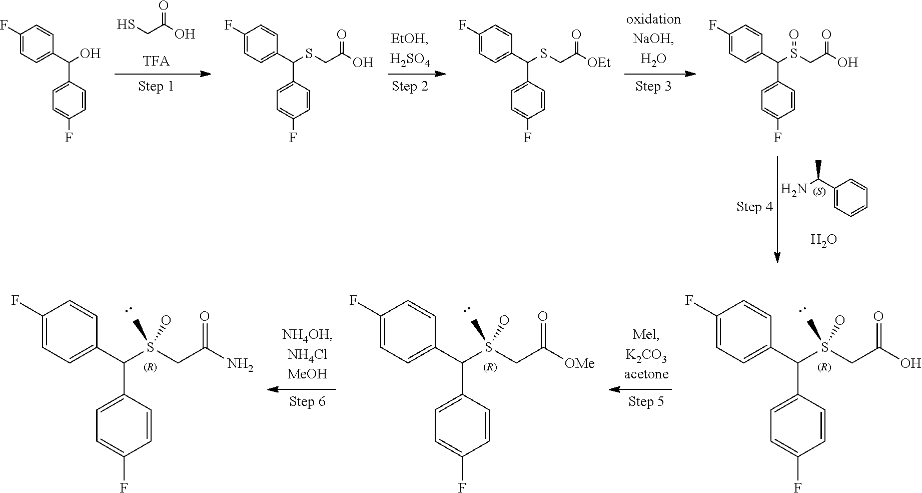

Lauflumide is tested at 10-5 M, calculated as a % inhibition of control specific binding of a radioactively labeled ligand specific for each target.

This binding profile panel was broadly defined with roughly an equal number of selective, central and peripheral therapeutically relevant targets, including native animal tissues, radioligands and specific enzymes involved in cell cycle regulation in accordance with Eurofins Standard Operating Procedure (www.eurofins.fr).

For radioligand binding experiments, the half maximal inhibitory concentration (IC₅₀) and the half maximal effective concentration (EC₅₀) values were determined (via computer software) by nonlinear regression analysis of the competition curves using Hill equation curve fitting. The inhibition constants (Kᵢ) were calculated using the Cheng-Prusoff equation (Kᵢ=IC₅₀/(1+(L/K_{D})), where L is the concentration of radioligand in the assay, and K_{D} is the affinity of the radioligand for the receptor.

The results are expressed as a % control specific binding ([measured specific binding/control specific binding]×100) and as a % inhibition of control specific binding (100−[(measured specific binding/control specific binding)×100]) obtained in the presence of the test compounds.

Results showing an inhibition (or stimulation) lower than 25% are not considered significant and mostly attributable to variability of the signal around the control level.

Low to moderate negative values have no real meaning and are attributable to variability of the signal around the control level.

An inhibition or stimulation of more than 50% is considered a significant effect of the test compounds and between 25% and 50% indicated of weak to moderate effects that should be confirmed by further testing as they are within a range where more inter-experimental variability can occur.

Fifty percent is a common cut-off for further investigation (i.e. determination of IC₅₀ or EC₅₀ values from concentration-response curves).

Principal significant or pertinent findings of these binding assays are respectively presented for Lauflumide in Table 1.

TABLE 1
Binding activity sites for Lauflumide

| | % Inhibition at 10⁻⁵M |
|---|---|
| Assay | Lauflumide |
| DAT (h) (antagonist radioligand) | 84.4 |
| Histamine 2 (h) (H2) (agonist radioligand) | 46.6 |
| NPY1 (h) (reverse agonist radioligand) | 27.3 |
| Relaxin-3 (RXFP3) (h) (agonist radioligand) | 23.9 |

Lauflumide selectively binds to the dopamine transporter (DAT) with a DA reuptake inhibition of 84.4% and thus possibly exerts some of its wake-promoting effect by disrupting the dopamine transport by DAT and hence raising extracellular concentrations of dopamine (DA), which results in wakefulness (Study 100014859 CEREP 20/03/14, unpublished data). Other potential effects on other pharmacological targets involved in wake enhancing, have been found on the binding profile of Lauflumide.

Lauflumide is found to weakly bind with RXFP3 receptors (Study US073-0006869-Q Eurofins/leadHunter 8/1/19; unpublished data) (Table 1).

In these assays' compounds were tested in agonist and antagonist mode with the GPCR Biosensor Assays and match to this design:

### Cell Handling

1. cAMP Hunter cell lines were expanded from freezer stocks according to standard procedures.

2. Cells were seeded in a total volume of 20 μL into white walled, 384-well microplates and incubated at 37° C. for the appropriate time prior to testing.

3. cAMP modulation was determined using the DiscoverX HitHunter cAMP XS+ assay.

### Gs Agonist Format

1. For agonist determination, cells were incubated with sample to induce response.

2. Media was aspirated from cells and replaced with 15 μL 2:1 HBSS/10 mM Hepes: cAMP XS+ Ab reagent.

3. Intermediate dilution of sample stocks was performed to generate 4×sample in assay buffer.

4. 5 μL of 4× sample was added to cells and incubated at 37° C. or room temperature for 30 or 60 minutes. Vehicle concentration was 1%.

### Gi Agonist Format

1. For agonist determination, cells were incubated with sample in the presence of EC80 forskolin to induce response.

2. Media was aspirated from cells and replaced with 15 μL 2:1 HBSS/10 mM Hepes: cAMP XS+ Ab reagent.

3. Intermediate dilution of sample stocks was performed to generate 4× sample in assay buffer containing 4× EC80 forskolin.

4. 5 μL of 4× sample was added to cells and incubated at 37° C. or room temperature for 30 or 60 minutes. Final assay vehicle concentration was 1%.

### Allosteric Modulation Format

1. For allosteric determination, cells were pre-incubated with sample followed by agonist induction at the EC20 concentration.

2. Media was aspirated from cells and replaced with 10 μL 1:1 HBSS/10 mM Hepes: cAMP XS+ Ab reagent.

3. Intermediate dilution of sample stocks was performed to generate 4× sample in assay buffer.

4. 5 μL of 4× compound was added to the cells and incubated at room temperature or 37° C. for 30 minutes.

5. 5 μL of 4×EC20 agonist was added to the cells and incubated at room temperature or 37° C. for 30 or 60 minutes. For Gi-coupled GPCRs, EC80 forskolin was included.

### Inverse Agonist Format (Gi Only)

1. For inverse agonist determination, cells were pre-incubated with sample in the presence of EC20 forskolin.

2. Media was aspirated from cells and replaced with 15 μL 2:1 HBSS/10 mM Hepes: cAMP XS+Ab reagent.

3. Intermediate dilution of sample stocks was performed to generate 4× sample in assay buffer containing 4× EC20 forskolin.

4. 5 μL of 4× sample was added to cells and incubated at 37° C. or room temperature for 30 or 60 minutes. Final assay vehicle concentration was 1%.

### Antagonist Format

1. For antagonist determination, cells were pre-incubated with sample followed by agonist challenge at the EC80 concentration.

2. Media was aspirated from cells and replaced with 10 μL 1:1 HBSS/Hepes: cAMP XS+ Ab reagent.

3. 5 μL of 4× compound was added to the cells and incubated at 37° C. or room temperature for 30 minutes.

4. 5 μL of 4×EC80 agonist was added to cells and incubated at 37° C. or room temperature for 30 or 60 minutes. For Gi coupled GPCRs, EC80 forksolin was included.

### Signal Detection

1. After appropriate compound incubation, assay signal was generated through incubation with L cAMP XS+ ED/CL lysis cocktail for

one hour followed by incubation with 20 μL cAMP XS+ EA reagent for three hours at room temperature.

2. Microplates were read following signal generation with a PerkinElmer Envision™ instrument for chemiluminescent signal detection.

### Data Analysis

1. Compound activity was analyzed using CBIS data analysis suite (ChemInnovation, CA).

2. For Gs agonist mode assays, percentage activity is calculated using the following formula:

% Activity=100%×(mean RLU of test sample−mean RLU of vehicle control)/(mean RLU of MAX control−mean RLU of vehicle control).

3. For Gs positive allosteric mode assays, percentage modulation is calculated using the following formula: http://www.eurofinsdiscoveryservices.com Confidential Jun. 25 2021

5% Modulation=100%×(mean RLU of test sample−mean RLU of EC20 control)/(mean RLU of MAX control−mean RLU of EC20 control).

4. For Gs antagonist or negative allosteric mode assays, percentage inhibition is calculated using the following formula: % Inhibition=100%×(1−(mean RLU of test sample−mean RLU of vehicle control)/(mean RLU of EC80 control−mean RLU of vehicle control)).

5. For Gi agonist mode assays, percentage activity is calculated using the following formula:

% Activity=100%×(1−(mean RLU of test sample−mean RLU of MAX control)/(mean RLU of vehicle control−mean RLU of MAX control)).

6. For Gi positive allosteric mode assays, percentage modulation is calculated using the following formula: % Modulation=100%×(1−(mean RLU of test sample−mean RLU of MAX control)/(mean RLU of EC20 control−mean RLU of MAX control)).

7. For Gi inverse agonist mode assays, percentage activity is calculated using the following formula: % Inverse Agonist Activity=100%×((mean RLU of test sample−mean RLU of EC20 forskolin)/(mean RLU of forskolin positive control−

mean RLU of EC20 control)).

8. For Gi antagonist or negative allosteric mode assays, percentage inhibition is calculated using the following formula: % Inhibition=100%×(mean RLU of test sample−mean RLU of EC80 control)/(mean RLU of forskolin positive control−mean RLU of

EC80 control).

For agonist and antagonist assays, data was normalized to the maximal and minimal response observed in the presence of control ligand and vehicle.

For Gi cAMP assays, the following forskolin concentration was used:
RXFP3 cAMP: 20 μM Forskolin
RXFP4 cAMP: 20 μM Forskolin

The following EC80 concentrations were used:
RXFP3 cAMP: 0.0003 μM Relaxin-3
RXFP4 cAMP: 0.01 μM Relaxin-3

### Effects of Lauflumide in a Rat Model of Severe Fatigue

In the following example, Lauflumide is also called NLS-4.

### Materials and Methods

Manipulations of animals were conducted carefully in order to reduce stress at the minimum. All the experiments were performed in compliance with the guidelines of the French Ministry of Agriculture for experiments with laboratory animals (law 2013-118).

Experiments were conducted in standard conditions (T°=22.0±1.5° C.), with artificial light in quiet conditions (no noise except those generated by ventilation and by the apparatus used for experiments).

Experiments were conducted blindly.

The animals have not been subjected to other experiments before the study.

### Animals

| | |
|---|---|
| Specie, strain, | Rats, Sprague-Dawley |
| sex | |
| Age | 7-8 weeks old at the time of testing |
| Number of | N = 88 |
| animals | |
| Origin (breeder) | Janvier Labs, France |
| Housing | Group housing (2 rats/cage): 1290D Eurostandard |
| | Type III cages (Tecniplast, Italy) in transparent |
| | polycarbonate (42.5 cm deep; 26.6 cm large; 15.5 |
| | cm high, area = 820 cm²). |
| | Cages are covered with a stainless steel grid in |
| | which food and a bottle are placed. A stainless steel |
| | removable divider separates food and water. |
| | Sleep deprived groups: see paragraph Erreur ! |
| | Source du renvoi introuvable. |
| Litter | Aspen Small (SDS Dietex, France) |
| Enrichment | Wood brick |
| Temperature | 22.0 ± 1.5° C. |
| Hygrometry | 50 ± 30% (not guaranteed; measured not controlled) |
| Air renewal | Fresh air, 12-25 vol/h |
| Lighting | 20-30 Lux |
| Day/night cycle | Reversed 12 h/12 h cycle; light off 9:00-21:00/on: |
| | 21:00-9:00 (except during the fatigue period, see |
| | paragraph Erreur ! Source du renvoi introuvable.. |
| Food | Rat-mouse A04 (Safe, France) available ad libitum |
| Drink | Tap water, available ad libitum |

### Drugs

| | |
|---|---|
| NLS-4 | Lauflumide |
| Vehicle | 5% Tween in distilled water |
| Administration route | p.o. |
| Doses studied | 16, 32, 64, 128, 256 mg/kg |
| Correction factor | 1 |
| (e.g. salt/base ratio) | |
| Number of applications | 3 |
| Application volume | 2 ml/kg of body weight. |
| Preparation | Prepared freshly before experiments. |
| Storage conditions during test | Ambient temperature (22-23° C.) |
| Modafinil | Modafinil |
| Vehicle | Tween in distilled water |
| Administration route | p.o. |
| Doses studied | 32, 64, 128 mg/kg |
| Correction factor | 1 |
| (e.g. salt/base ratio) | |
| Number of applications | 3 |
| Preparation | Prepared freshly before experiments. |
| Storage conditions during test | Ambient temperature (22-23° C.) |

### Sleep Deprivation Procedure

Animals are individually housed in transparent polycarbonate cages (1354G Eurostandard Type IV cage, Tecniplast, Italy, 595×380×200 mm, floor area=1820 cm²) filled with water (23±1° C.) to a height of 1.5 cm. A platform (5 cm height, 6.5 cm diameter) was located at the center of the cage. Animals can stay on the platform in order to stay dry but falls from it when it falls in deep sleep. This procedure has been shown to induce sleep—and especially REM sleep—deprivation. During this period, animals were placed in a modified light/dark cycle with variable durations of the light and dark periods which change every day. This procedure is intended to disrupt the circadian rhythm.

The Sham group was housed in normal condition for the same period under a 12/12 h light/dark cycle.

Animals were weighed every day.

### Recording of Circadian Activity

After the end of the fatigue period, animals were placed in transparent polycarbonate cages (Type E, Iffa Credo, France, 450×300×200 mm, floor area=1032 cm²), under a 12 h/12 h cycle. Cages were covered with a stainless steel grid in which food and a bottle are placed. A stainless steel removable divider separated food and water. The cages were placed in infrared photobeam detection systems (Acti-track, LSI Leticca, Panlab) allowing to record continuously the distance travelled in the cage. Locomotor activity was recorded continuously for 3 days. The test started at the beginning of the dark phase of the light/dark cycle.

### Groups

Seventy-six animals were subjected to the fatigue procedure and to the tests as described above. Twelve animals (Sham group) were housed in standard condition.

After the end of the Fatigue procedure, animals were divided into 10 groups which received before tests (total: 3 administrations) a per-os (p.o., oral gavage) administration of:
Sham (no fatigue) group (N=12): Vehicle.
Control group (N=11): Vehicle.
Modafinil 32 (N=10): modafinil (32 mg/kg).
Modafinil 64 (N=10): modafinil (64 mg/kg).
Modafinil 128 (N=5): modafinil (128 mg/kg).
NLS-4 16 (N=5): NLS-4 (16 mg/kg).
NLS-4 32 (N=10): NLS-4 (32 mg/kg).
NLS-4 64 (N=10): NLS-4 (64 mg/kg).
NLS-4 128 (N=10): NLS-4 (128 mg/kg).
NLS-4 256 (N=5): NLS-4 (256 mg/kg).

### Data Analysis

Read-out: distance travelled during 24-h periods, 12-h dark and light periods and during 2-h periods.

Data are expressed as mean and standard error of mean (SEM).

Statistical analyses: Student's t test, repeated measure ANOVA.

A difference is considered statistically significant at p≤0.05.

### Results

### Circadian Activity

### Effect of Sleep Deprivation

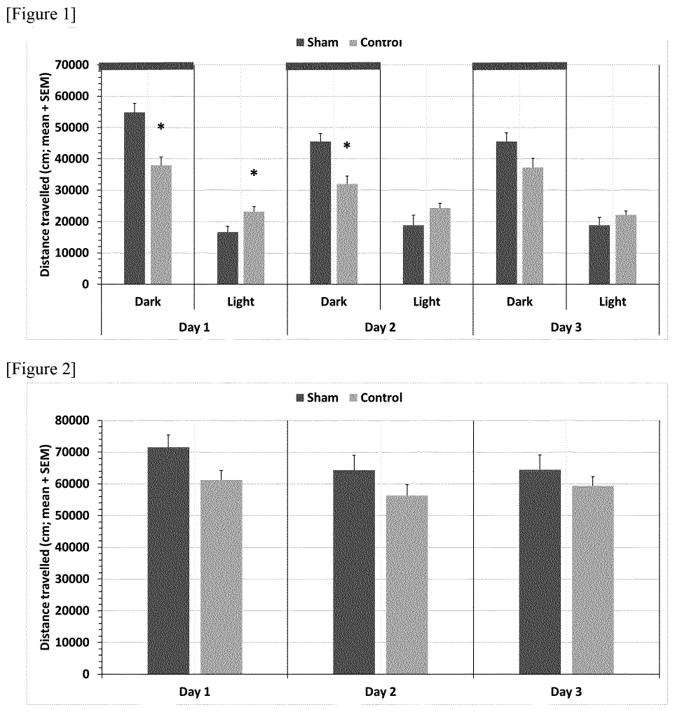

As shown in FIG. 1:
The distance travelled during the dark phase was lower for the Control group than for the Sham group on days 1 and 2 and was not significantly different between groups on day 3.
The distance travelled during the light phase was higher for the Control group than for the Sham group on day 1, and was not significantly different between groups on days 2 and 3.

As shown in FIG. 2:
The total distance travelled during the two phases (full 24-h period) tended to be lower for the Control group than for the Sham group on day 1 and was not significantly different between the Control group and the Sham group on days 2 and 3.

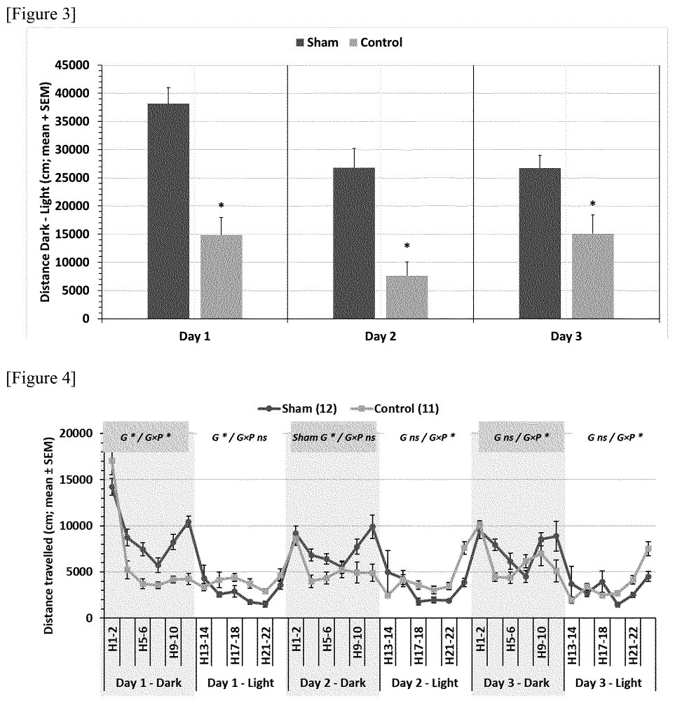

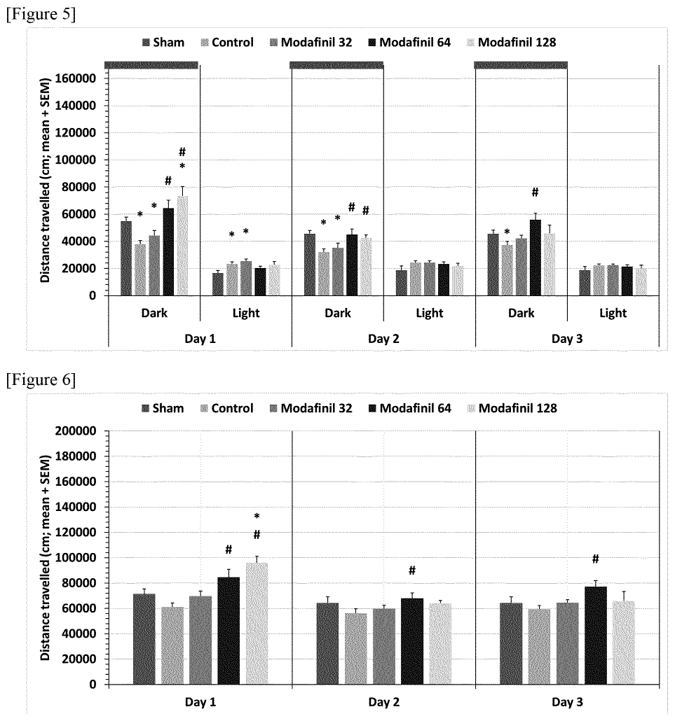

As shown in FIG. 3:
The difference of distance travelled between the dark phase and the light phase was lower for the Control group than for the Sham group on days 1, 2 and 3.

The distance travelled during each 2-h period of the dark and light phases is shown in FIG. 4.

On day 1, the repeated measure ANOVAs show that:
During the dark phase, the total distance travelled was lower for the Control group than for the Sham group as mentioned above (significant group effect), and the change in locomotor activity during the 12-h period was different between groups (significant group×period interaction).
During the light phase, the total distance travelled was higher for the Control group than for the Sham group as mentioned above (significant group effect), but the change in locomotor activity during the 12-h period was not significantly different between groups (group×period interaction: ns).

On day 2, the repeated measure ANOVAs show that:
During the dark phase, the total distance travelled was lower for the Control group than for the Sham group as mentioned above (significant group effect), but the change in locomotor activity during the 12-h period was not significantly different between groups (group×period interaction: ns).
During the light phase, the total distance travelled was not significantly different between groups as mentioned above (group effect: ns), but the change in locomotor activity during the 12-h period was different between groups (significant group×period interaction).

On day 3, the repeated measure ANOVAs show that:
During the dark phase, the total distance travelled tended to be lower for the Control group than for the Sham group as mentioned above (close to significant group effect), and the change in locomotor activity during the 12-h period was different between groups (significant group×period interaction).
During the light phase, the total distance travelled was not significantly different between groups as mentioned above (group effect: ns), but the change in locomotor activity during the 12-h period was different between groups (significant group×period interaction).

Conclusion. These findings show that the fatigue procedure induced an impairment in the circadian activity, that is a decrease in motor activity during the dark period (when rats are normally active) and an increase in motor activity during the period of light (when rats are largely inactive). This impairment partially declined over time but was still present 3 days after the end of the fatigue procedure.

### Effect of Modafinil

As shown in Erreur! Source du renvoi introuvable, the distance travelled by rats treated with modafinil during the dark and light phases:
Was not significantly different between the Modafinil 32 group and the Control group during both the dark and light periods on days 1, 2 and 3.
Was higher for the Modafinil 64 group than for the Control group during the dark period and was not significantly different between these two groups during the light period on days 1, 2 and 3.
Was higher for the Modafinil 128 group than for the Control group during the dark period on days 1 and 2 only and was not significantly different between these two groups during the light period on days 1, 2 and 3; however, it should be noted that Modafinil (128 mg/kg) also increase the distance travelled in comparison with the Sham group during the dark phase on day 1.

As shown in FIG. 6:
The total distance during the full 24-h period:
   Was not significantly different between the Modafinil 32 group and the Control on days 1, 2 and 3.
   Was higher for the Modafinil 64 group than for the Control group on days 1, 2 and 3.
Was higher for the Modafinil 128 group than for the Control group on day 1 only; however, it should be noted that Modafinil (128 mg/kg) also increase the distance travelled in comparison with the Sham group on day 1.

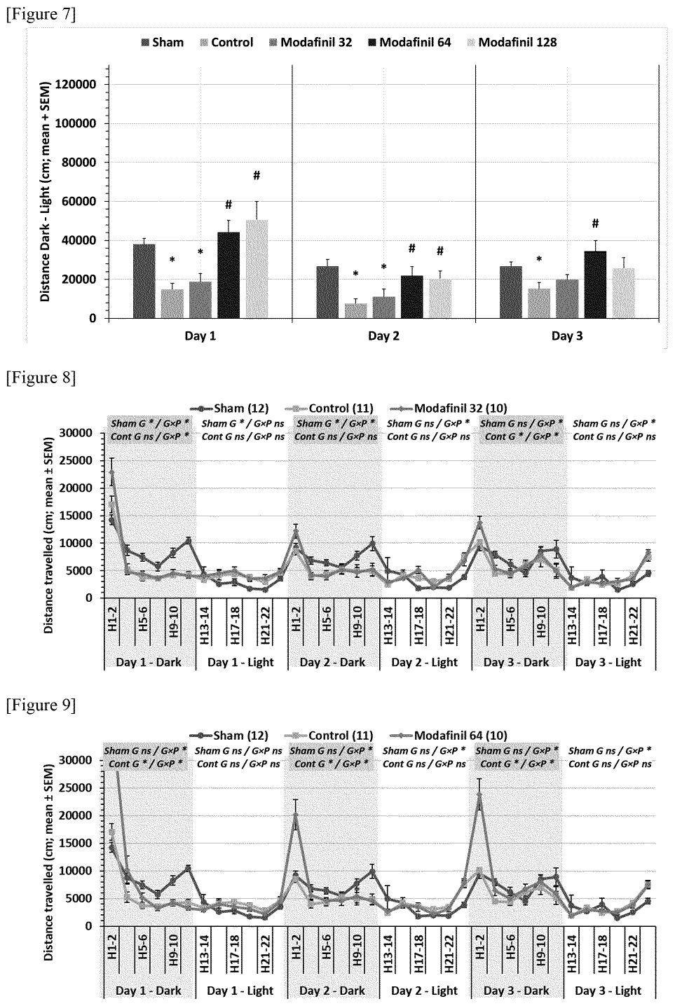

As shown in FIG. 7:
The difference of distance travelled between the dark phase and the light phase:
   Was not significantly different between the Modafinil 32 group and the Control on days 1, 2 and 3.
   Was higher for the Modafinil 64 group than for the Control group on days 1, 2 and 3.
   Was higher for the Modafinil 128 group than for the Control group on days 1 and 2 only.

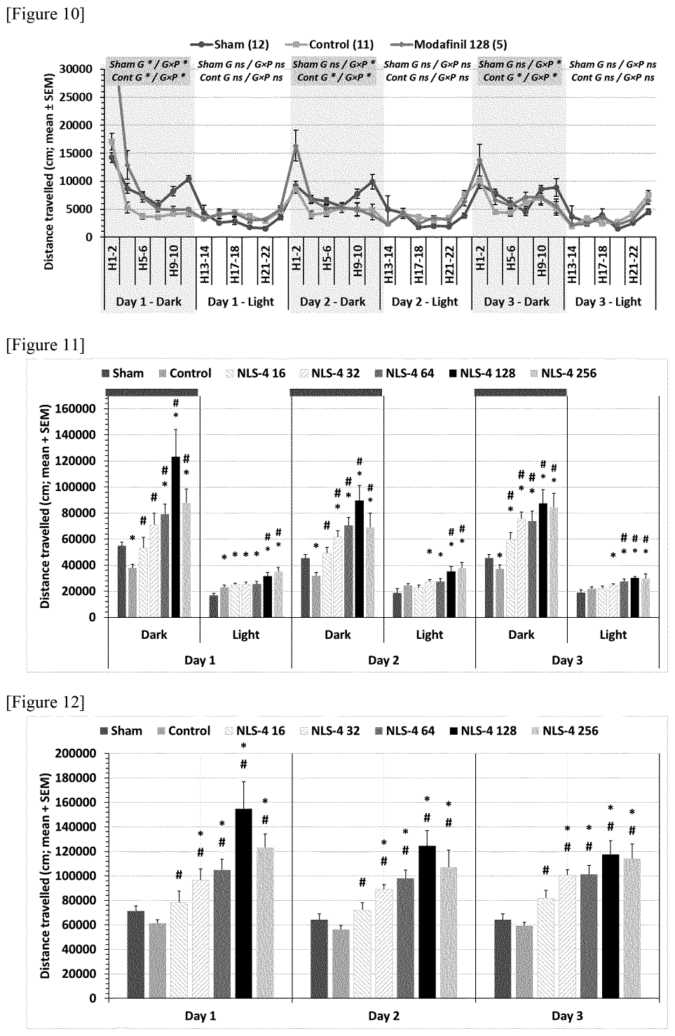

The distance travelled during each 2-h period of the dark and light phases is shown in FIG. 8, FIG. 9 and FIG. 10. These results show that Modafinil can increase the distance travelled during the dark phase and induced less effect on activity during the light period, as mentioned above.

Conclusion. These findings show that Modafnil (64, 128 mg/kg) induced an increase in locomotor activity which may reduce the impairment of circadian rhythm induced by the fatigue procedure.

### Effect of Lauflumide (NLS-4)

As shown in Erreur! Source du renvoi introuvable, the distance travelled by rats treated with NLS-4 during the dark and light phases:
Was higher for the NLS-4 16 group than for the Control group during the dark phase and was not significantly different between the NLS-4 16 group and the Control group during the light phase on days 1, 2 and 3; however, it should be noted that NLS-4 (16 mg/kg) also increased the distance travelled in comparison with the Sham group during the light phase on day 1 and during the dark phase on day 3.
Was higher for the NLS-4 32 group than for the Control group during the dark phase and was not significantly different between the NLS-4 32 group and the Control group during the light phase on days 1, 2 and 3; however, it should be noted that NLS-4 (32 mg/kg) also increased the distance travelled in comparison with the Sham group during the light phase on days 1, 2 and 3, and during the dark phase on days 2 and 3.
Was higher for the NLS-4 64 group than for the Control group during the dark phase on days 1, 2 and 3 and during the light phase on day 3 only; it should be noted that NLS-4 (64 mg/kg) also increased the distance travelled in comparison with the Sham group during both the dark and light phase on days 1, 2 and 3.
Was higher for the NLS-4 128 group than for the Control group during both the dark phase and the light phase on days 1, 2 and 3; it should be noted that NLS-4 (128 mg/kg) also increased the distance travelled in comparison with the Sham group during both the dark and light phase on days 1, 2 and 3.
Was higher for the NLS-4 256 group than for the Control group during both the dark phase and the light phase on days 1, 2 and 3; it should be noted that NLS-4 (256 mg/kg) also increased the distance travelled in comparison with the Sham group during both the dark and light phase on days 1, 2 and 3.

As shown in FIG. 12:
The total distance travelled during the full 24-h period was higher for the NLS-4 16, 32, 64, 128 and 256 groups than for the Control group on days 1, 2 and 3; however, it should be noted that NLS-4 (32-256 mg/kg) also increased the distance travelled in comparison with the Sham group during days 1, 2 and 3.

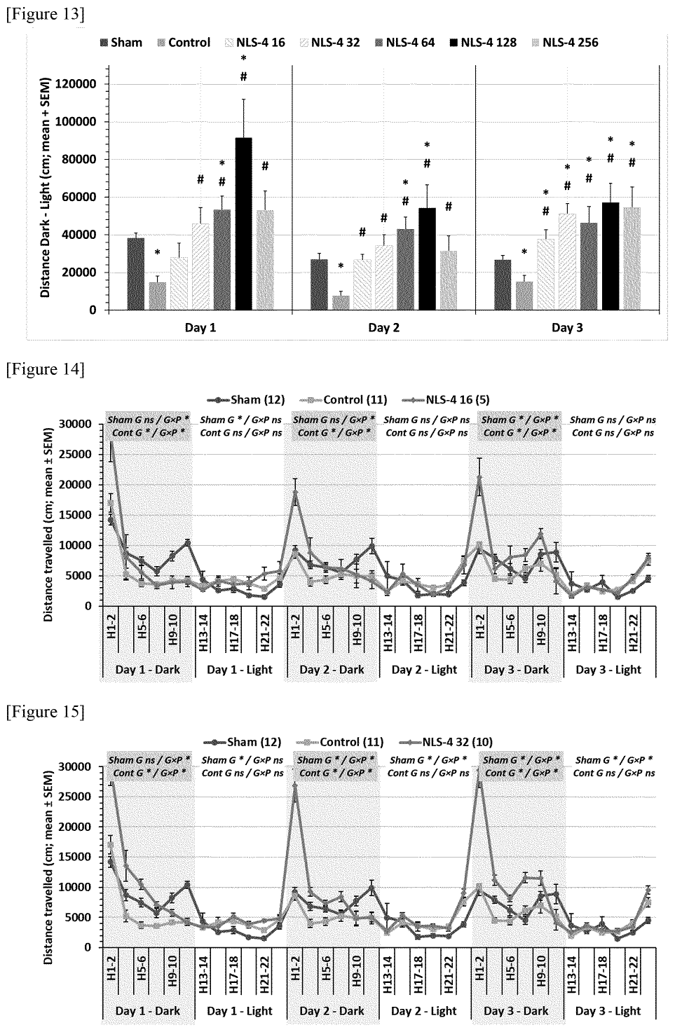

As shown in FIG. 13:
The difference of distance travelled between the dark phase and the light phase was higher or tended to be higher for the NLS-4 16, 32, 64, 128 and 256 groups than for the Control group on days 1, 2 and 3; however, it should be noted that NLS-4 (16-256 mg/kg) also increased the distance travelled in comparison with the Sham group during day 3 only (at 16, 32 and 256 mg/kg) and on days 1, 2 and 3 (at 64 and 128 mg/kg).

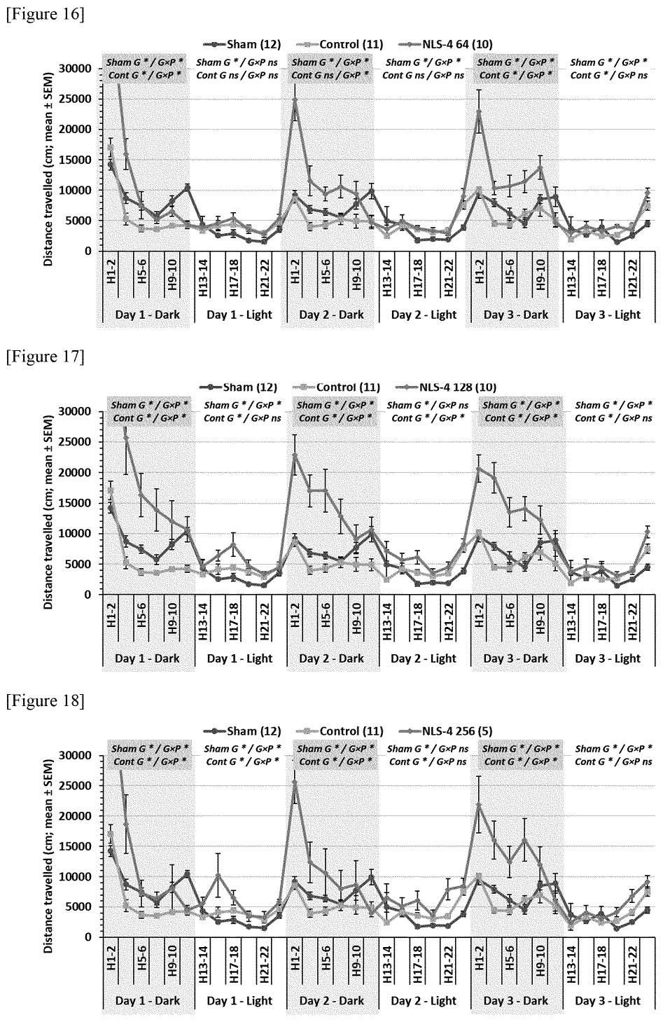

The distance travelled during each 2-h period of the dark and light phases is shown in FIG. 14, FIG. 15, FIG. 16, FIG. 17 and FIG. 18. These results show that NLS-4 dose-dependently increased the distance travelled during the dark phase and induced less effect on activity during the light period, as mentioned above, but also that NLS-4 dramatically increased the distance travelled during the first 2-h period (at 16 mg/kg), during the 8-h period (at 32 mg/kg) and during the 10-h period (at 64-256 mg/kg), after administration.

Conclusion. These findings show that NLS-4 (16-256 mg kg) induced an increase in locomotor activity which may reduce the impairment of circadian rhythm induced by the fatigue procedure.

Comparison of NLS-4 vs. Modafinil

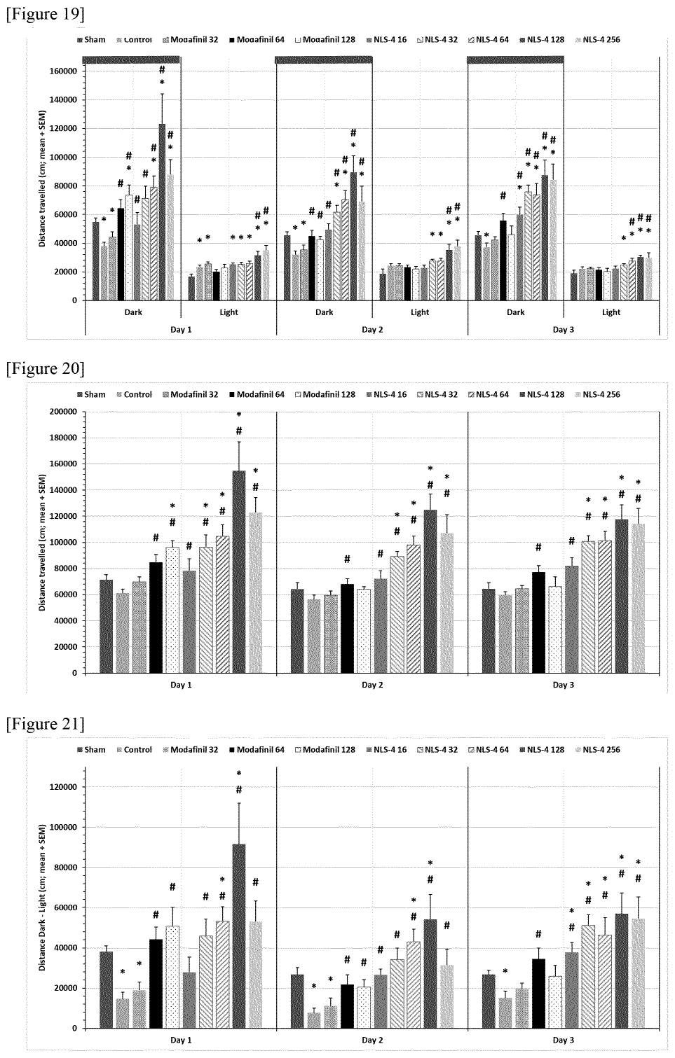

The distance travelled by rats from all groups during the dark and light phases are shown in FIG. 19.

As mentioned above, the total distance travelled during the full 24-h period was significantly increased by modafinil (128 mg/kg) and by NLS-4 (32, 64, 128 and 256 mg/kg) (FIG. 20). Furthermore, as shown in FIG. 21, the difference of distance travelled between the dark phase and the light phase was dramatically decreased by the fatigue procedure, showing an impairment of circadian activity. The difference of distance travelled between the dark phase and the light phase was increased in comparison with the Control (Fatigue) group by Modafinil (64, 128 mg/kg) and by NLS-4 at all doses.

Modafinil and NLS-4 at higher doses have a high significant motor stimulant effect. Therefore, we focused comparison of NLS-4 vs. Modafinil groups to NLS-4 16, Modafinil 32 and Modafinil 64 groups.

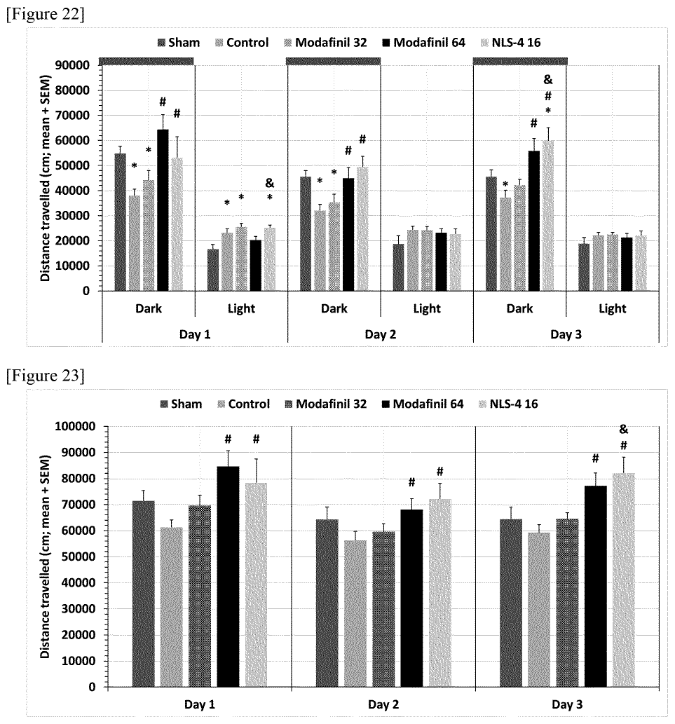

FIG. 22 show that the distance travelled during the dark and light phases:
For the Modafinil 32 group:
   Was not significantly different from the Control group during both the dark and light periods on days 1, 2 and 3.
   Was lower than the Sham group during the dark phase on days 1 and 2 only and was higher than the Sham group during the light phase on day 1 only.
For the Modafinil 64 group:
   Was higher than the Control group during the dark period on days 1, 2 and 3 and was not significantly different from the Control group during the light phase on days 1, 2 and 3.
   Was not significantly different from the Sham group during both the dark and light periods on days 1, 2 and 3.
For the NLS-4 16 group:
   Was higher than the Control group during the dark period on days 1, 2 and 3 and was also higher from the Control group during the light period on day 1 only.
   Was higher than the Sham group during the dark period on day 3 only and was higher than the Control group during the light periods on day 1 only.
   Was higher than the Modafinil 32 group during the dark period on day 3 only and during the light period on day 1 only.
   Was not significantly different from the Modafinil 64 group during both the dark and light periods on days 1, 2 and 3.

As shown in FIG. 23, the total distance during the full 24-h period:
For the Modafinil 32 group:
   Was not significantly different from both the Control group and the Sham group on days 1, 2 and 3.
For the Modafinil 64 group:
   Was higher than the Control group days 1, 2 and 3.
   Was not significantly different from the Sham group on days 1, 2 and 3.
For the NLS-4 16 group:
   Was higher than the Control group days 1, 2 and 3.
   Was not significantly different from the Sham group on days 1, 2 and 3.
   Was higher than the Modafinil 32 group on day 3 only.
   Was not significantly different from the Modafinil 64 group on days 1, 2 and 3.

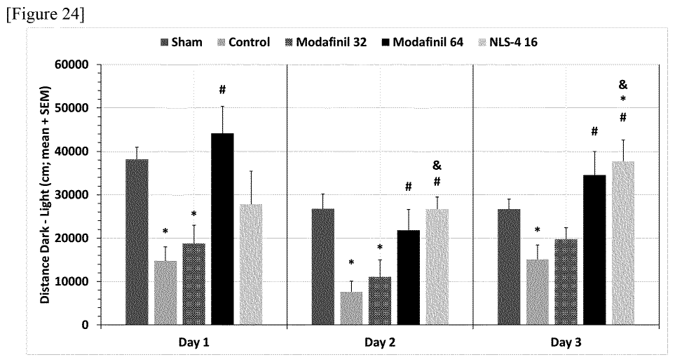

As shown in FIG. 24, the difference of distance travelled between the dark phase and the light phase:
For the Modafinil 32 group:
   Was not significantly different from the Control group on days 1, 2 and 3.
   Was not lower than the Sham group on days 1 and 2 only.
For the Modafinil 64 group:
   Was higher than the Control group days 1, 2 and 3.
   Was not significantly different from the Sham group on days 1, 2 and 3.
For the NLS-4 16 group:
   Was higher or tended to be higher than the Control group days 1, 2 and 3.
   Was higher than the Sham group on day 3 only.
   Was higher than the Modafinil 32 group on days 2 and 3 only.
   Was not significantly different from the Modafinil 64 group on days 1, 2 and 3.

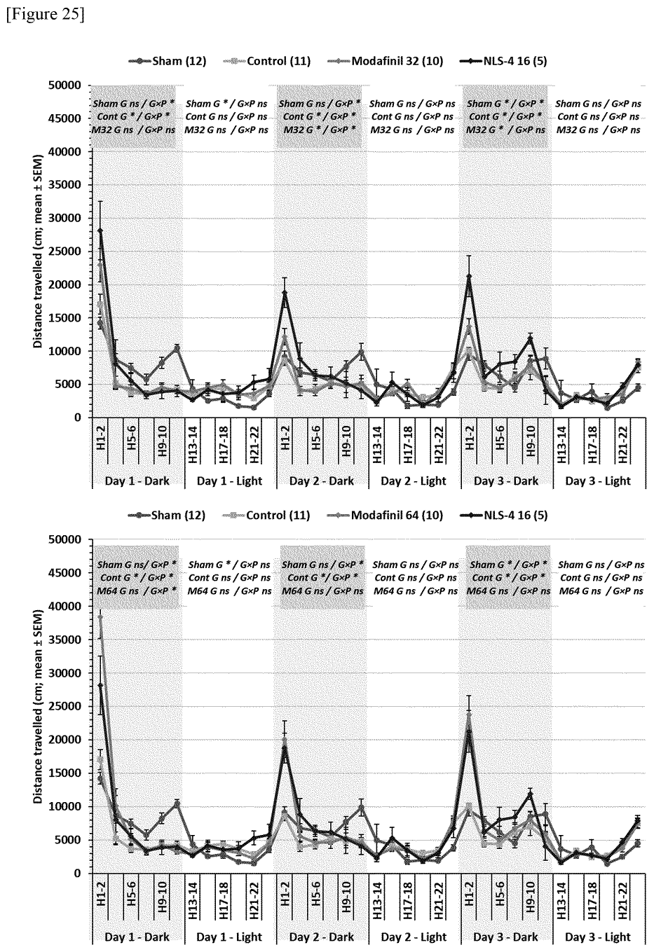

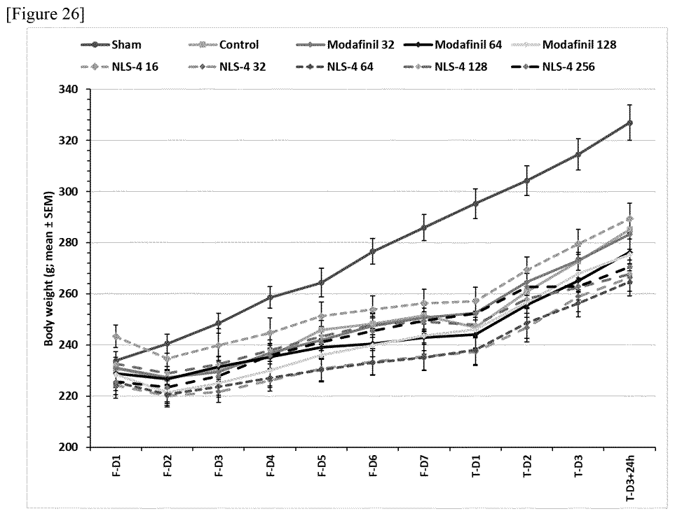

The distance travelled during each 2-h period of the dark and light phases is shown in FIG. 25. These results show that Modafinil (32, 64 mg/kg) and NLS-4 (16 mg/kg) increased motor activity during the first 2-h periods following the dosing. Modafinil (32 mg/kg) induced few or no modification of the activity during the following 22 h. The increase in motor activity induced by Modafinil (64 mg/kg) and NLS-4 (16 mg/kg) was more sustainable, especially on days 2 and 3 and tended to be more pronounced for NLS-4 (16 mg/kg) than for modafinil (64 mg/kg). NLS-4 16 and Modafinil 64 groups were not statistically different, except a significant group×period interaction which was significantly different on day 1, as a result of a higher hyperactivity in the Modafinil 64 group than in the NLS-4 16 group during the first 2-h period following the first dosing. On the other hand, the NLS-4 16 group was significantly different from the Modafinil 32 group—i.e. was more active—during the dark periods on days 2 and 3.

Conclusion. These findings show that NLS-4 (16 mg/kg) induced an increase in locomotor activity which was of the same magnitude to that of modafinil (64 mg kg) and which was significantly higher to that induced by modafinil (32 mg/kg). Therefore, these results suggest that NLS-4 may reduce the impairment of circadian rhythm induced by the fatigue procedure at dose four times lower than modafinil.

### Body Weight

The fatigue procedure induced a decrease in the body weight. The weight growth curve returned to normal and the weight loss remained constant during the test period, after discontinuation of the fatigue procedure. The weight loss was not modified by modafinil (32-128 mg/kg) and by NLS-4 (16 mg/kg) but was aggravated by NLS-4 (32-256 mg/kg).

### BIBLIOGRAPHY

1. Tenforde M W, Kim S S, Lindsell C J, et al. Symptom Duration and Risk Factors for Delayed Return to Usual Health Among Outpatients with COVID-19 in a Multistate Health Care Systems Network—United States, March-June 2020. MMWR Morb Mortal Wkly Rep. 2020; 69(30):993-998. doi:10.15585/mmwr.mm6930el
2. Hellwig S, Domschke K. [Post-COVID syndrome-Focus fatigue]. Nervenarzt. Published online May 23, 2022:1-7. doi:10.1007/s00115-022-01306-1
3. Huang B, Niu Y, Zhao W, Bao P, Li D. Reduced Sleep in the Week Prior to Diagnosis of COVID-19 is Associated with the Severity of COVID-19. Nat Sci Sleep. 2020; 12:999-1007. doi:10.2147/NSS.S263488
4. Huang C, Huang L, Wang Y, et al. 6-month consequences of COVID-19 in patients discharged from hospital: a cohort study. Lancet. 2021; 397(10270):220-232. doi:10.1016/S0140-6736(20)32656-8
5. Premraj L, Kannapadi N V, Briggs J, et al. Mid and long-term neurological and neuropsychiatric manifestations of post-COVID-19 syndrome: A meta-analysis. J Neurol Sci. 2022; 434:120162-120162. doi:10.1016/j.jns.2022.120162
6. Alwan N A, Johnson L. Defining long COVID: Going back to the start. Med (N Y). 2021; 2(5):501-504. doi:10.1016/j.medj.2021.03.003
7. Renaud-Charest O, Lui L M W, Eskander S, et al. Onset and frequency of depression in post-COVID-19 syndrome: A systematic review. Journal of Psychiatric Research. 2021; 144:129-137. doi:10.1016/j.jpsychires.2021.09.054
8. Benitez I D, Moncusi-Moix A, Vaca R, et al. Sleep and Circadian Health of Critical COVID-19 Survivors 3 Months After Hospital Discharge. Crit Care Med. 2022; 50(6):945-954. doi: 10.1097/CCM.0000000000005476
9. Boiko D I, Skrypnikov A M, Shkodina A D, Hasan M M, Ashraf G M, Rahman M H. Circadian rhythm disorder and anxiety as mental health complications in post-COVID-19. Environ Sci Pollut Res Int. 2022; 29(19):28062-28069. doi:10.1007/s11356-021-18384-4
10. Ceban F, Ling S, Lui L M W, et al. Fatigue and cognitive impairment in Post-COVID-19 Syndrome: A systematic review and meta-analysis. Brain, Behavior, and Immunity. 2022; 101:93-135. doi:10.1016/j.bbi.2021.12.020
11. McCarthy M J. Circadian rhythm disruption in Myalgic Encephalomyelitis/Chronic Fatigue Syndrome: Implications for the post-acute sequelae of COVID-19. Brain, Behavior, & Immunity—Health. 2022; 20:100412. doi:10.1016/j.bbih.2022.100412
12. Wong T L, Weitzer D J. Long COVID and Myalgic Encephalomyelitis/Chronic Fatigue Syndrome (ME/CFS)—A Systemic Review and Comparison of Clinical Presentation and Symptomatology. Medicina (Kaunas). 2021; 57(5). doi:10.3390/medicina57050418
13. Fukuda K, Straus S E, Hickie I, Sharpe M C, Dobbins J G, Komaroff A. The chronic fatigue syndrome: a comprehensive approach to its definition and study. International Chronic Fatigue Syndrome Study Group. Ann Intern Med. 1994; 121(12):953-959. doi:10.7326/0003-4819-121-12-199412150-00009
14. Reeves W C, Lloyd A, Vernon S D, et al. Identification of ambiguities in the 1994 chronic fatigue syndrome research case definition and recommendations for resolution. BMC Health Serv Res. 2003; 3(1):25. doi:10.1186/1472-6963-3-25
15. Beyond Myalgic Encephalomyelitis Chronic Fatigue Syndrome: Redefining an Illness. National Academies Press (US); 2015. doi:10.17226/19012
16. Lam M H B, Wing Y K, Yu M W M, et al. Mental morbidities and chronic fatigue in severe acute respiratory syndrome survivors: long-term follow-up. Arch Intern Med. 2009; 169(22):2142-2147. doi:10.1001/archinternmed.2009.384
17. Moldofsky H, Patcai J. Chronic widespread musculoskeletal pain, fatigue, depression and disordered sleep in chronic post-SARS syndrome; a case-controlled study. BMC Neurol. 2011; 11:37. doi:10.1186/1471-2377-11-37
18. Komaroff A L, Bateman L. Will COVID-19 Lead to Myalgic Encephalomyelitis/Chronic Fatigue Syndrome?FrontMed (Lausanne). 2020; 7:606824. doi:10.3389/fmed.2020.606824
19. Toogood P L, Clauw D J, Phadke S, Hoffman D. Myalgic encephalomyelitis/chronic fatigue syndrome (ME/CFS): Where will the drugs come from?Pharmacol Res. 2021; 165:105465. doi:10.1016/j.phrs.2021.105465
20. Advisory Panel Members Worry About Potential for Abuse of Drug That's Similar to GHB. FDA Panel Rejects Xyrem as Fibromyalgia Treatment. www.webmd.com/fibromyalgia/news/20100820/fda-panel-rejects-xyrem-as-fibromyalgia-treatment
21. A Trial of Solriamfetol in the Treatment of Myalgic Encephalomyelitis/Chronic Fatigue Syndrome. https://clinicaltrials.gov/ct2/show/NCT04622293
22. Fletcher M A, Rosenthal M, Antoni M, et al. Plasma neuropeptide Y: a biomarker for symptom severity in chronic fatigue syndrome. Behav Brain Funct. 2010; 6:76. doi:10.1186/1744-9081-6-76
23. Morales-Medina J C, Dumont Y, Benoit C E, et al. Role of neuropeptide Y Y₁ and Y₂ receptors on behavioral despair in a rat model of depression with co-morbid anxiety. Neuropharmacology. 2012; 62(1):200-208. doi:10.1016/j.neuropharm.2011.06.030
24. Giovannini M G, Efoudebe M, Passani M B, et al. Improvement in fear memory by histamine-elicited ERK2 activation in hippocampal CA3 cells. J Neurosci. 2003; 23(27):9016-9023. doi:10.1523/JNEUROSCI.23-27-09016.2003
25. Gianlorenço A C L, Serafim K R, Canto-de-Souza A, Mattioli R. Emotional memory consolidation impairment induced by histamine is mediated by H1 but not H2 receptors. Brain Res Bull. 2012; 89(5-6):197-202. doi:10.1016/j.brainresbull.2012.09.003
26. Silva-Marques B, Gianlorenço A C L, Mattioli R. Intracerebellar vermis histamine facilitates memory consolidation in the elevated T maze model. Neurosci Lett. 2016; 620:33-37. doi:10.1016/j.neulet.2016.03.010
27. Milgram N W. Adrafinil: A novel vigilance promoting agent. CNS Drug Review. 1999; 5(3):193-212.
28. Lucas I, Mulraney M, Sciberras E. Sleep problems and daytime sleepiness in children with ADHD: Associations with social, emotional, and behavioral functioning at school, a cross-sectional study. Behav Sleep Med. 2019; 17(4):411-422. doi:10.1080/15402002.2017.1376207
29. Wisor J P, Nishino S, Sora I, Uhl G H, Mignot E, Edgar D M. Dopaminergic role in stimulant-induced wakefulness. J Neurosci. 2001; 21(5):1787-1794. doi:10.1523/JNEUROSCI.21-05-01787.2001
30. Wisor J P, Dement W C, Aimone L, Williams M, Bozyczko-Coyne D. Armodafinil, the R-enantiomer of modafinil: wake-promoting effects and pharmacokinetic profile in the rat. Pharmacol Biochem Behav. 2006; 85(3):492-499. doi:10.1016/j.pbb.2006.09.018
31. Bobak M J, Weber M W, Doellman M A, et al. Modafinil Activates Phasic Dopamine Signaling in Dorsal and Ventral Striata. J Pharmacol Exp Ther. 2016; 359(3):460-470. doi:10.1124/jpet.116.236000
32. Dunn D, Hostetler G, Iqbal M, et al. Wake-promoting agents: search for next generation modafinil: part II. Bioorg Med Chem Lett. 2012; 22(6):2315-2317. doi:10.1016/j.bmcl.2012.01.064
33. Turner C, Belyavin A J, Nicholson A N. Duration of activity and mode of action of modafinil: Studies on sleep and wakefulness in humans. J Psychopharmacol. 2014; 28(7):643-654. doi:10.1177/0269881113508173
34. Mereu M, Bonci A, Newman A H, Tanda G. The neurobiology of modafinil as an enhancer of cognitive performance and a potential treatment for substance use disorders. Psychopharmacology (Berl). 2013; 229(3):415-434. doi:10.1007/s00213-013-3232-4
35. Mereu M, Chun L E, Prisinzano T E, Newman A H, Katz J L, Tanda G. The unique psychostimulant profile of (±)—modafinil: investigation of behavioral and neurochemical effects in mice. Eur J Neurosci. 2017; 45(1):167-174. doi:10.1111/ejn.13376
36. Rambert F A, Pessonnier J, de Sereville J E, Pointeau A M, Duteil J. [A unique psychopharmacologic profile of adrafinil in mice]. J Pharmacol. 1986; 17(1):37-52.
37. Bernardi R E, Broccoli L, Spanagel R, Hansson A C. Sex differences in dopamine binding and modafinil conditioned place preference in mice. Drug Alcohol Depend. 2015; 155:37-44. doi:10.1016/j.drugalcdep.2015.08.016
38. Wuo-Silva R, Fukushiro D F, Borgoi A R, et al. Addictive potential of modafinil and cross-sensitization with cocaine: a pre-clinical study. Addict Biol. 2011; 16(4):565-579. doi:10.1111/j.1369-1600.2011.00341.x
39. Bayard S, Langenier M C, Dauvilliers Y. Effect of psychostimulants on impulsivity and risk taking in narcolepsy with cataplexy. Sleep. 2013; 36(9):1335-1340. doi:10.5665/sleep.2958
40. Ma S, Smith C M, Blasiak A, Gundlach A L. Distribution, physiology and pharmacology of relaxin-3/RXFP3 systems in brain. Br J Pharmacol. 2017; 174(10):1034-1048. doi:10.1111/bph.13659
41. Ma S, Gundlach A L. Ascending control of arousal and motivation: role of nucleus incertus and its peptide neuromodulators in behavioural responses to stress. J Neuroendocrinol. 2015; 27(6):457-467. doi:10.1111/jne.12259
42. Smith C M, Shen P J, Banerjee A, et al. Distribution of relaxin-3 and RXFP3 within arousal, stress, affective, and cognitive circuits of mouse brain. J Comp Neurol. 2010; 518(19):4016-4045. doi:10.1002/cne.22442
43. Voglsanger L M, Read J, Ch'ng S S, et al. Differential Level of RXFP3 Expression in Dopaminergic Neurons Within the Arcuate Nucleus, Dorsomedial Hypothalamus and Ventral Tegmental Area of RXFP3-Cre/tdTomato Mice. Front Neurosci. 2020; 14:594818. doi:10.3389/fnins.2020.594818

1-9. (canceled)

10. A method of prevention and/or treatment of chronic fatigue syndrome comprising the administration of a compound of formula (I) 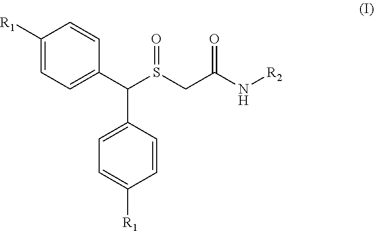
with R₁=a halogen atom selected in the group consisting of F, Cl, Br, and I, and R₂=H or OH,
or a pharmaceutically acceptable isomer, salt, and/or solvate thereof, to a patient in need thereof.

11. The method of claim 10, wherein the compound of formula (I) is 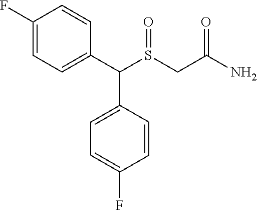 [SMILES: NC(=O)CS(=O)C(c1ccc(F)cc1)c1ccc(F)cc1]
or a pharmaceutically acceptable isomer, salt, and/or solvate thereof.

12. The method of claim 10, wherein a therapeutic dose of 50 mg/day to 500 mg/day is administered to the patient in need thereof.

13. The method of claim 12, wherein the therapeutic dose is 80 mg/day to 320 mg/day.

14. The method of claim 10, wherein the chronic fatigue syndrome is a post-viral syndrome.

15. The method of claim 14, wherein the chronic fatigue syndrome is a long COVID condition.

16. A method of prevention and/or treatment of chronic fatigue syndrome comprising the administration of a pharmaceutical composition comprising a compound of formula (I) as defined in claim 10 or a pharmaceutically acceptable isomer, salt, and/or solvate thereof and a pharmaceutically acceptable excipient.

17. The method of claim 16, wherein the compound of formula (I) is 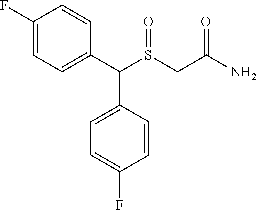 [SMILES: NC(=O)CS(=O)C(c1ccc(F)cc1)c1ccc(F)cc1]
or a pharmaceutically acceptable isomer, salt, and/or solvate thereof.

18. The method of claim 16, wherein the chronic fatigue syndrome is a post-viral syndrome.

19. The method of claim 18, wherein the chronic fatigue syndrome is a long COVID condition.

20. The method of claim 16, wherein the pharmaceutical composition is suitable for oral or parenteral administration.

21. The method of claim 16, wherein the pharmaceutical composition is in the form of a solution, a tablet, a capsule, or a Transdermal Delivery System.

22. The method of claim 16, wherein the pharmaceutical composition is in the form of an injectable solution.
